(19) United States
(12) Patent Application Publication
(10) Pub. No.: US 2011/0015600 A1
(43) Pub. Date: Jan. 20, 2011

(54) Lower cost and environmentally effective, personal adjusting reusable diaper insert and fold lock pouch

(76) Inventor: Tuyetnhung T. Pham, Saugus, MA (US)

Correspondence Address: Tuyetnhung T. Pham, 110 Golden Hills Road, Saugus, MA, 01906-4004, US

(21) Appl. No.: 12/460,155

(22) Filed: Jul. 15, 2009

(51) Int. Cl. A61F 13/53; D05B 23/00; B32B 7/08

(52) U.S. Cl. 604/367; 604/385.15; 112/475.09; 156/92

(57) ABSTRACT

The diaper insert is formed from multiple uneven panels attached together in the middle providing comfort without requiring folding while using less drying time and energy. The diaper pouch is formed from rectangular panels and has a fold lock. Since pieces are rectangular, it can be be both adapted to a continuous feed process utilizing equipment available from other industries to increase productivity, and be arranged to minimize waste in the manufacturing process. The cover panel and fold lock are composed of woven fabric laminated with polyurethane. Since polyurethane does not absorb water, this helps shorten drying time. The pocket panel is made from woven fabric that can last for many wash and dry cycles. Personal fitting and adjusting for different size diaper inserts can be managed by making two of the sides adjustable length by utilizing either stretchable or non-stretch cords with stoppers.

### BACKGROUND OF THE INVENTION

1. Field of the Invention

This invention relates to reusable diapers with a focus on making them both more cost-effective and environmentally-friendly for both manufacturers and consumers. It also addresses fitting adjustments for personal comfort.

2. General Background

There has been a long debate over the merits of reusable versus disposable diapers. Reusable diaper support groups claim that they save the environment by reducing solid fill and favoring cottons (or other natural fiber products) over the plastics typically used by disposable diapers. They can be potentially used by multiple children within the same family, reducing waste still further. Disposable diaper support groups claim that they save the environment by reducing the energy required in washing and drying, and the reduced use of fertilizers and pesticides needed to grow cotton (or other natural fibers) needed to make reusable diapers.

This invention addresses the perceived weaknesses of the reusable diapers by both filling the need for a lower-cost reusable diaper manufacturing process with less environmental impact, and filling the need for a reusable diaper with a shorter and less expensive washing/drying cycle with less energy consumption.

The current shape and complexity of reusable diapers requires human operators on sewing machines resulting in very high manufacturing cost and nigh impossibility of automation. Some disposable diaper supporters claim that the energy required to produce a single disposable diaper is less than the energy to wash and dry a reusable diaper twenty times. If true, clearly this is not really the way to save energy. This invention reduce the energy required to wash and dry a diaper, and paves the way to bringing the production cost of a single reusable diaper down much closer to the cost of a single disposable diaper.

Most current reusable diapers and reusable diaper covers have a waterproof layer usually coated on top of cotton or a natural fiber like bamboo or hemp. Besides (normally) requiring chemicals to grow the relevant plants, the resulting combination takes more energy to dry than synthetic fibers like microfiber, polyester, etc., and by virtue of the synthetic waterproof layer tends to lose most of the desired properties of the natural fiber (like airiness and absorbency) anyway.

Each of the types of reusable diapers currently used have problems. The all-in-one pocket style and fitted shape insert style diapers consume a great deal of energy to wash and dry. The pre-fold style diapers take quite a bit of time to fold prior to use. This invention addresses both of these problems.

None of the current fitted reusable diapers have a fold lock to prevent waste from running down due to an extended period lying down in bed like this invention does.

Most of the fitted reusable diapers feature elastic bands to perform some automatic fitting, but none allow the consumer additional adjustment to individual body proportions like this invention does. Current reusable diapers have the same thickness of absorbent material at the waste and crotch; this is not really necessary for comfort or absorbing ability and wastes energy washing and drying. This invention makes it easy to add absorbent material to areas that need it at the expense of those areas that do not.

3. Prior Art

As already discussed, none of the current reusable diapers adequately balance manufacturing expense, washing and drying expense, environmental impact, ease-of-use, and wearer comfort. Many patents cover individual aspects in a different manner from this invention. Some noteworthy ones include the following:

U.S. Pat. No. 4,548,604 (Ellsworth) focuses on wearer comfort and is fabricated from a double layer of cloth, having an elastic member attached to the outer edge and arranged metal snaps to adjust to different sized wearers. This design is difficult to fabricate. It also takes more time and energy to dry. As U.S. Pat. No. 5,185,011 (Strasser) provides multiple layers sewn together it is again hard to dry.

U.S. Pat. No. 4,681,581 (Coates) and U.S. Pat. No. 6,623,467 (Charles-Lundaahl) utilize a folded rectangular sheet. Obviously a rectangular sheet is easy and quick to dry but it needs extra time to fold and prepare before use. Charles-Lundaahl's folding method is especially difficult and hard to follow with many steps.

U.S. Pat. No. 4,576,601 (Brain) defines an open plastic pair of pants to hold a liner. It provides easy access for changing but it is impractical to produce as it needs a human to perform each task. Likewise U.S. Pat. No. 6,579,273 (Dupuy) with its hourglass shape and multiple snaps will not work well with continuos feeds making it expensive to produce.

U.S. Pat. No. 5,160,331 (Forester) provides an environmentally sound absorbent insert with a continuous feed process that uses paper and/or biodegradable material. It is better for environment than disposable diapers but due to its use of paper material has a much shorter usable life than this invention.

### SUMMARY OF THE INVENTION

This invention provides a reusable diaper design that saves energy and time in a washing and drying cycle by employing a single layer waist band diaper pouch and a non-folded multiple layer diaper insert that may be opened like a book when drying.

This invention also provides a reusable diaper design optimized to minimize waste material in a production process that can be readily adapted to a continuous feed process.

Finally, this invention provides a reusable diaper design that improves upon the comfort of existing reusable diapers by allowing for personal fitting adjustments, reducing leaks while the wearer is lying down, simplifying fastening with direction-neutral One-Wrap technology fasteners, and enhancing tummy support.

### BRIEF DESCRIPTION OF THE DRAWINGS

For a further understanding of the invention, references should be made to the detailed descriptions provided in conjunction with the accompanying drawings, in which, like parts are given like reference numerals, and wherein:

FIGS. 1a, b, c, d are views of diaper insert embodiment;

FIGS. 2a, b, c, d, e are views of diaper pouch embodiment;

FIG. 3 is an inside-out view of the diaper pouch embodiment;

FIG. 4 is a view of the process of assembling a single diaper pouch embodiment;

FIGS. 5a, b, c are views of the process of making reusable diaper pouches via a continuous feed process.

FIGS. 6a, b, c, d, e, f, g are views of the process of making reusable diaper inserts via a continuous feed process.

### DETAILED DESCRIPTIONS OF PREFERRED EMBODIMENT

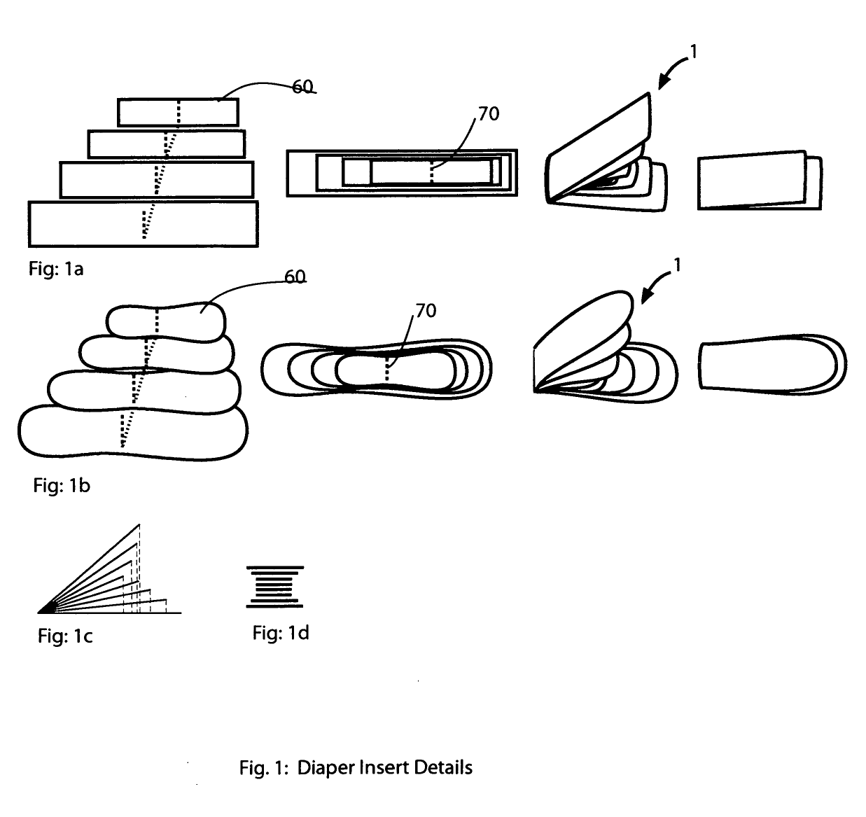

The diaper insert as illustrated in FIG. 1a show top views of absorbent panels 60 in both roughly rectangular and figure eight shapes FIG. 1b arranged unevenly with stitches 70 near the middle. FIG. 1c is the front view of a diaper insert and FIG. 1d the side view.

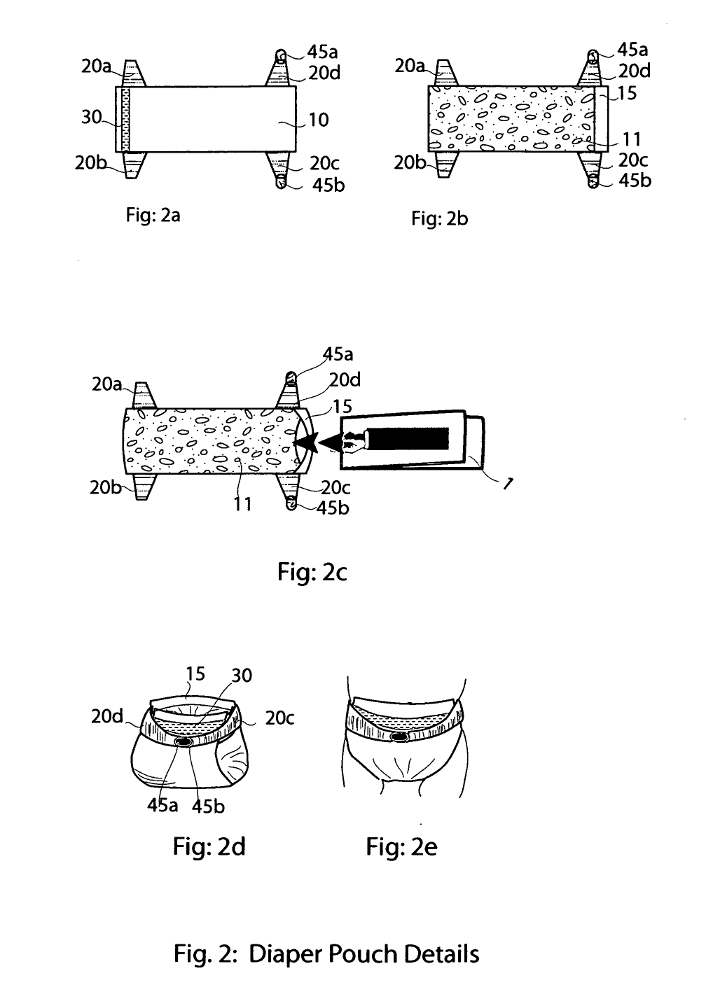

The diaper pouch as illustrated in FIG. 2a shows top views of the pouch, where the corner flaps 20a, 20b, 20c, 20d are attached in between pocket panel 10 and cover panel 11 (in the top view we just see the cover panel 11). Two ONE-WRAP® straps 45a, 45b are attached to back corner flaps 20c, 20d.

The tummy support band 30 lines up between the front corner flaps 20a, 20b.

The diaper pouch as illustrated in FIG. 2b has bottom views of the pouch where the four corner flaps 20a, 20b, 20c, 20d are attached in between pocket panel 10 and cover panel 11 (in the bottom view we just see the pocket panel 10). Fold lock panel 15 encapsulates the inserted diaper.

The diaper pouch and insert as illustrated in FIG. 2c show the direction to insert the insert diaper to diaper pouch via the open entry of fold lock panel 15.

The pouch and insert diaper as illustrated in FIG. 2d show corner flaps 20c and 20d serve like waist support conjunction with straps fasteners 45a and 45b closed in front of tummy support 30. It also shows the fold lock in the back 15. FIG. 2e show the diaper pouch and insert in human body.

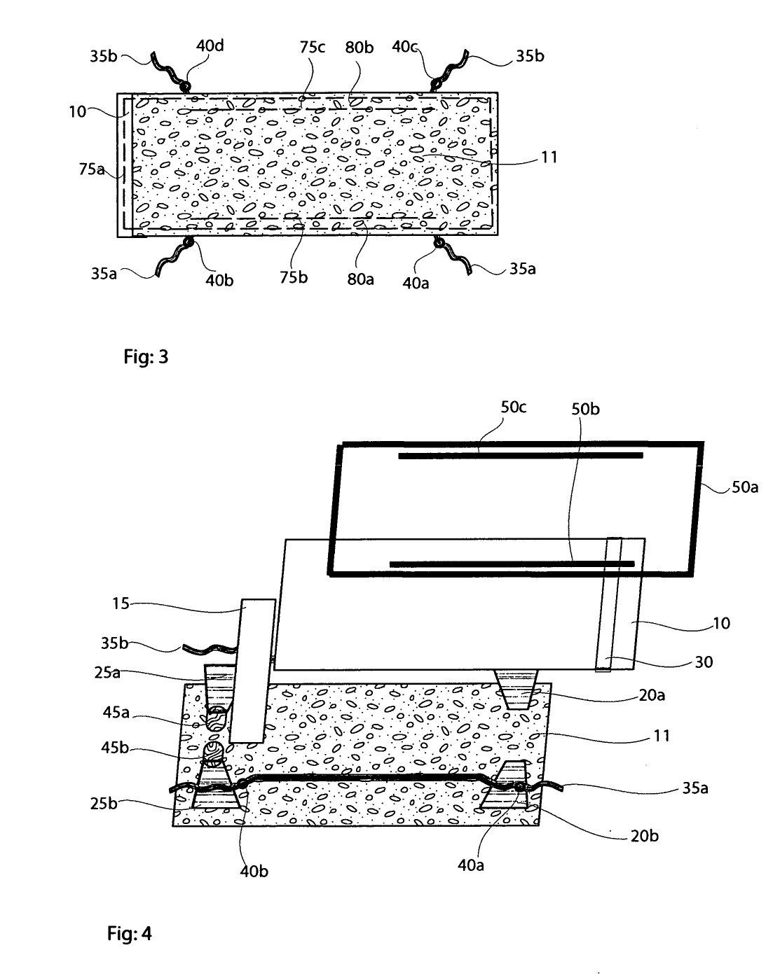

FIG. 3 is the view of the pouch diaper inside-out, the attaching line 75a binding pouch panel 10, cover panel 11, and fold lock panel 15 to form the pouch, attaching lines 75b, 75c parallel with the longer sides of the pouch provide turners 80a and 80b for cords 35a and 35b accordingly in place, while stoppers 40a, 40b can move along cord 35a and stoppers 40c, 40d can move along cord 35b for fitting adjustment.

FIG. 4 shows the assembly of a diaper pouch. Starting with cover panel 11, front corner flaps 20a, 20b, 20c, 20d are put on top of the four corners, cords 35a, 35b are assembled with stoppers 40a, 40b, 40c, 40d along the longer sides of cover panel 11, the fold lock panel 15 is put on the back corner flaps parallel with the shorter sides of cover panel 11, and the pocket panel is finally put on the top. The fixture 50a, 50b, 50c will create attaching lines 75a, 75b, 75c accordingly.

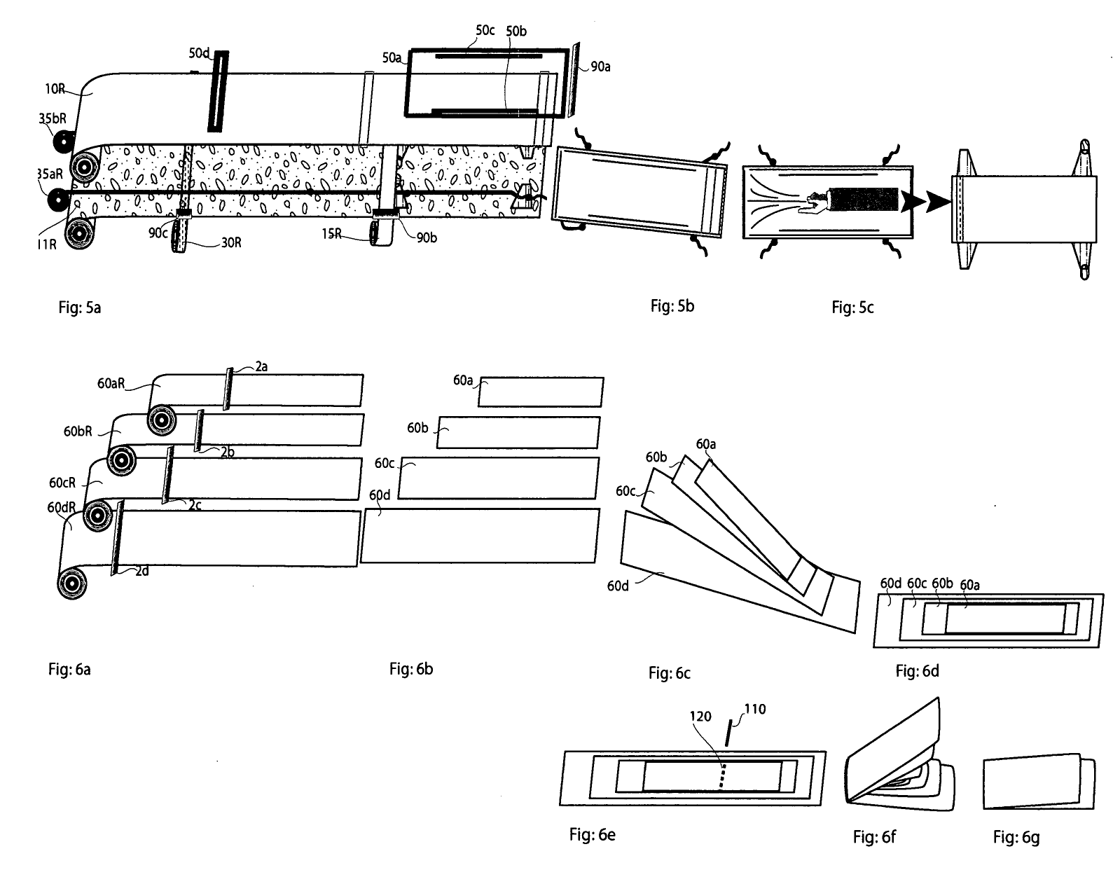

FIG. 5a shows the process of making reusable diaper pouches via a continuous feed process. Rolls of pocket panel 10R, cover panel 11R, pre-assembled stopper and cord 35aR and 35bR are fed continuously at the same time and rate; rolls of tummy support band 30R and fold lock panel 15R are fed continuously at the same time and rate, while corner flaps are placed in between the roll of pocket panel 10R and the roll of cover panel 11R. The fixtures 50a, 50b, 50c, 50d will perform attaching lines 75a, 75b, 75c and tummy support band accordingly. The knife 90a will cut rolls of pocket panel 10R, cover panel 11R, pre-assembled stopper and cord 35aR and 35bR. The knives 90b, 90c will cut rolls of tummy support band 30R and fold lock panel 15R accordingly. Speed of knives go up and down so as to synchronize with the speed of roll feeding. FIG. 5b shows a reusable pouch inside-out with its cover panel face up. The last step in the process is to turn the diaper pouch rightside-out as show in FIG. 5c.

FIG. 6a shows the process of making a reusable diaper insert via a continuous feed process. Multiple rolls of difference size insert panel feed in with difference rate as figure show four rolls 60aR, 60bR, 60cR, 60dR, its will be cut to difference length by knives 2a, 2b, 2c, 2d accordingly Speed of knives go up and down so as to synchronize with the speed of rolls feeding. FIG. 6b show all insert panel are cut in length, then it is transported by covey belt as FIG. 6c and piled up as show at FIG. 6d. The fixture 110 will create attaching lines 120 as FIG. 6e. Insert will be fold in middle as FIG. 6f.

What is claimed is as follows:

1. A reusable diaper insert composed of multiple panels attached together so as to be able to open like a book.

2. A reusable diaper pouch formed from a rectangular cover panel, a rectangular pocket panel, and a rectangular fold lock panel.

3. A reusable diaper insert according to claim 1 where the individual panels are of different size and/or shape and/or reusable material.

4. A reusable diaper insert according to claim 1 where the individual panels are arranged unevenly or attached together off center.

5. A reusable diaper pouch according to claim 2 where the two parallel sides of each panel have adjustable cords with or without stoppers.

6. A reusable diaper pouch according to claim 2 utilizing corner flaps with or without straps.

7. A reusable diaper pouch according to claim 2 with a tummy support band.

8. A reusable diaper pouch according to claim 2 wherein said cover panel and fold lock panel utilize polyurethane film with or without flame-retardant, stain-resistant additions laminated onto woven textile.

9. A reusable diaper pouch according to claim 2 wherein said pocket panel is formed from woven textile.

10. A reusable diaper pouch according to claim 5 wherein said cords are formed from stretchable or non-stretchable washable material.

11. A reusable diaper pouch according to claim 5 wherein knots or a cord lock serve as stopper.

12. A reusable diaper pouch according to claim 6 wherein the corner flaps are formed from stretchable woven textiles.

13. A reusable diaper pouch according to claim 6 wherein the straps employ One-Wrap technology.

14. A reusable diaper pouch according to claim 7 wherein the tummy support band is knitted high-pile loop textile.

15. The process of manufacturing the reusable pocket diaper insert of claim 1 via a continuous feed.

16. The process of manufacturing the reusable pocket diaper pouch of claim 2 via a continuous feed.

17. The attaching of components of the reusable pocket diaper insert of claim 1 being performed by traditional sewing machine, programmable pattern sewing machine and/or any equipment using heat application such as heating elements or ultrasonic/radio frequency heat generation.

18. The attaching of components of the reusable pocket diaper pouch of claim 2 being performed by traditional sewing machine, programmable pattern sewing machine and/or any equipment using heat application such as heating elements or ultrasonic/radio frequency heat generation.
